(19) United States
(12) Patent Application Publication
(10) Pub. No.: US 2004/0209240 A1
(43) Pub. Date: Oct. 21, 2004

(54) Anti-neoplastic viruses

(75) Inventors: Richard Iggo, Lausanne (CH); Maddalena Malerba, Lausanne (CH)

Correspondence Address: NIXON & VANDERHYE, PC, 1100 N GLEBE ROAD, 8TH FLOOR, ARLINGTON, VA, 22201-4714, US

(73) Assignee: BTG International Limited, London (GB)

(21) Appl. No.: 10/647,111

(22) Filed: Aug. 25, 2003

Related U.S. Application Data
(63) Continuation-in-part of application No. PCT/GB03/03657, filed on Aug. 20, 2003.

(30) Foreign Application Priority Data
Aug. 22, 2002 (GB) 0219619.4

(51) Int. Cl. C12Q 1/70; C07H 21/04

(52) U.S. Cl. 435/5; 435/69.3; 435/320.1; 435/235.1; 435/325; 536/23.72

(57) ABSTRACT

A viral DNA construct encoding for a parvovirus that is capable of replication in a human or animal tumour cell is provided. The viral DNA consturct having one or more selected transcription factor binding sites operatively positioned such as to promote expression of open reading frames encoding non-structural viral proteins, wherein the selected transcription factor binding sites are for a transcription factor the level or activity of which is increased in a human or animal tumour cell relative to that of a normal human or animal cell of the same type.

### BACKGROUND

The present invention relates to viral agents that have application in the treatment of neoplasms such as tumours, particularly tumours derived from colon cells, more particularly liver tumours that are metastases of colon cell primary tumours. Still more particularly the invention relates to autonomous parvovirus constructs that selectively replicate in response to transcription activators present in tumour cells, these factors being present either exclusively or at elevated levels in tumour cells as compared to other cells, and thus cause tumour cell death and cell lysis.

Viruses which replicate selectively in tumour cells have great potential for gene therapy for cancer as they can spread progressively through a tumour until all of its cells are destroyed. Most current targeting strategies exploit tumour-specific defects in the regulation of cellular DNA replication or transcription. This overcomes the need to infect all tumour cells at the time the virus is injected, which is a major limitation to conventional replacement gene therapy, because in principle virus goes on being produced, lysing cells on release of new virus, until no tumour cells remain. An important fundamental distinction in cancer gene therapy is thus between single hit approaches, using non-replicating viruses, and multiple hit approaches, using replicating viruses.

Several replicating adenoviruses have now been tested in clinical trials for cancer (2). The strength of this approach is that the therapeutic effect of the injected virus is augmented by that of virus produced within the tumour. Since the acute side effects of treatment are directly related to the amount of virus injected (36), the ability to inject a smaller amount of virus is an important advantage of replicating viruses. Deliberate release of replicating viruses designed to kill human cells carries the obvious risk of starting new epidemics, but with adenovirus the immune system limits the number of cycles of virus replication so effectively that poor efficacy is a much more important barrier to the widespread use of these viruses for cancer therapy. The general strategy pursued with adenovirus has been first to attenuate the virus, ideally in a manner that confers tumour specificity, and only then to broaden the tropism or express toxic genes. Many such viruses are now in development, but all share the same basic adenovirus structure. The genetic instability of tumours is almost certain to produce broad spectrum resistance to these agents, at least in a minority of patients. To overcome this resistance it would be useful to have alternative agents drawn from an entirely different virus family.

Among the other DNA viruses available for the development of cancer therapeutics, most are known or suspected to cause serious diseases, such as progressive multifocal leucoencephalopathy in AIDS patients with JC virus or mesothelioma with SV40. Other DNA viruses are so large that the consequences of modification of the viral genome are difficult to predict. These viruses, like herpes viruses, often produce latent infections which would be a source of concern in cured patients. Still other DNA viruses are either difficult to produce, like papilloma viruses, or replicate in the cytoplasm, like pox viruses.

Of the autonomous parvoviruses B19 is the only virus known to cause human disease. Other parvoviruses, including MVM and H1, have a number of properties that make them interesting candidates for development as cancer therapeutics. They have been shown to reduce the incidence of spontaneous and chemically induced tumours in laboratory animals (13, 17, 40). They also possess intrinsic oncotropism and show selective toxicity to tumour cells in culture (7, 8, 40). Parvovirus particles are small, which should favour spread within tumours (14). Parvoviruses such as H1 and MVM are rodent parvoviruses that do not infect humans naturally. Hence there is no pre-existing antibody to these viruses and no reason to expect cross-resistance to adenovirus.

The drawbacks of autonomous parvoviruses are their inability to induce S phase and their limited potential for expressing transgenes. Although they are currently difficult to produce in large quantities, intensive efforts to produce AAV for gene therapy are likely to overcome this obstacle in the near future. Despite their inherent advantages, H1 and MVM are too restricted in their tropism to be useful without modification. For example, MVM decapsidation is blocked in some cells. Analysis of MVM host range mutants has identified blocks at multiple levels including decapsidation, amplification and post-encapsidation in human cell lines (29, 34)

The parvovirus family is divided into erythrovirus, for example B19 that can cause anaemia and stillbirth in humans; dependoviruses such as AAV, which can only replicate with help from another virus; and autonomous parvoviruses such as MVM, Hi, which are able to replicate without any help from another virus. Autonomous parvoviruses replicate autonomously in rapidly dividing cells. The genomes of autonomous parvoviruses apparently do not integrate, at least at a detectable level, into the host genome.

Autonomous parvovirus genomes are single-stranded DNA molecules about 5 kilobases (kb) in size. The genomes are organised such that the NS gene encoding the non-structural polypeptides NS1, multifunctional phosphoprotein (9), and NS2 is located on the left side of the genome and the VP genes encoding the structural polypeptides required for capsid formation are on the right side of the genome. Expression of the non-structural polypeptides (NS1 and NS2) is controlled by a transcription control sequence called P4 in most parvoviruses, which is located at about map unit position 4 of the genome, after the 3′ hairpin, (assuming the entire genome is 100 map units and numbering is from left to right).

The P4 promoter contains binding sites for E2F, Ets and Sp1 transcription factor, in addition to a TATA box.

Expression of the structural polypeptides is controlled by a transcription control sequence called P38, P39 or P40 in most parvoviruses, which is located at about map unit position 38 to about 40, depending on the autonomous parvovirus. NS1 serves as a trans-activator of the latter transcription control sequence (12, 20, 23). NS1 is also essential for virus replication (32) and appears to be the primary mediator of parvovirus cytotoxicity (5), particularly against tumour cells. Autonomous parvovirus genomes also have inverted repeat sequences (ITRs) at each end which contain essential signals for replication and encapsidation of the virus.

The small size of the virus means that it is difficult to express transgenes from replication competent parvoviruses. Hence, cell killing must rely on the intrinsic properties of the virus. Several toxic effects of parvoviruses have been described. The viral hairpins (ITRs) may trigger checkpoints which selectively kill p53 mutant cells (31, 37) and as mentioned above expression of the viral protein NS1 is also toxic, particularly to tumour cells (5). In appropriate circumstances, parvoviruses are perfectly capable of destroying whole lineages of cells, as for example B19 does when it produces aplastic anaemia. It is therefore desirable, as with all therapeutic viruses, that parvovirus replication and expression of toxic viral genes is restricted to target cells.

Artificially removing barriers to replication of wild type parvoviruses raises biosafety concerns. Previous attempts to restrict autonomous parvovirus replication to target cells were confounded by positive feedback of NS1 on its own expression (24). This feedback loop exists because NS1 binding sites are present in the P4 promoter and NS1 has a potent transactivation domain (22). Previous studies on the phH1 (H1/MVM) virus with a BglII linker replacing the E2F site in the P4 promoter showed that E2F activity is essential for virus production (11). Exogenous NS1 was able to complement the defect in trans, showing that the mutation did not interfere with other essential functions.

U.S. Pat. No. 5,585,254 discloses a large number of possible parvovirus based gene therapy vectors and proposes the use of a “modified P4 transcription control element that exhibits an enhanced or reduced ability to activate transcription of coding regions” (see col. 9 and examples 8 & 9). Specifically, U.S. Pat. No. 5,585,254 describes the construction of a LuIII recombinant parvovirus vector pPRE-Lu which is produced by removing the P4 promoter region of the LuIII infectious clone pGLu883 and replacing it with two copies of the PRE consensus sequence combined with a minimal promoter such as the TATA box plus cap site. The NS gene is operatively linked to the PRE response element. The PRE response element was selected because of the abundance of progesterone receptors in certain breast cancer cells. The aim of such a construct appears to be to link expression of toxic NS gene products to binding of progestin at the PRE response element.

WO 00/56909, incorporated herein by reference, describes adenoviruses that replicate in response to activation of tumour specific transcription factors, particularly of the wnt signalling pathway. These viruses have Tcf binding sites inserted into the E2 promoter, which regulates expression of the viral replication genes. Wnt signalling is pathologically activated in virtually all colon tumours, and to a lesser extent in other tumour types such as melanoma, which leads to transcription from promoters containing Tcf binding sites. The constitutive activation of the wnt pathway is caused by mutations in the APC, axin and β-catenin genes, thus inhibiting GSK-3β phosphorylation of β-catenin and its subsequent degradation by the proteosome (34). Cytoplasmic β-catenin enters the nucleus, where it can associate with members of the Tcf/Lef family of transcription factors and activate transcription of wnt target genes, such as c-myc, cyclin D1, Tcf1 and matrilysin. In addition to Tcf there are other transcription factors whose activity is known to increase in tumours. For example, RBPJκ and Gli-1, representing the endpoints of the notch and hedgehog signal transduction pathways, and HIF1alpha. The hedgehog pathway is activated by mutations in the patched and smoothened proteins in basal cell cancer. Notch mutations occur in some leukaemias. Telomerase activation is one of the hallmarks of cancer and results from increased activity of the telomerase promoter, although the mechanism is unknown.

The present inventors have determined that by inserting tumour specific transcription factor binding sites, particularly Tcf, into the P4 promoter region of a parvovirus, positive feedback by NS1 is prevented and transcription of P4 is tightly repressed in normal cells. This is possibly due to the presence of a threshold level of NS1 below which positive feedback is not triggered. Recruitment of basal levels of transcription factor to the promoter represses P4 transcription in normal cells and thereby maintains NS1 levels below this threshold.

The inventors have addressed the biosafety and specificity issues of using replicating parvoviruses by first attenuating the virus and including an additional layer of safety. They have inserted binding sites for Tcf family transcription factors into the P4 promoter, which controls expression of the NS1 and NS2 proteins. Tcf regulation of NS1 expression restricts virus replication to colon tumours having constitutive activation of the wnt signalling pathway, which is a universal causal oncogenic defect in colon tumours and also occurs in other tumour types (33). In normal cells, Tcfs recruit Groucho and other co-repressors to prevent transcription. Activation of wnt signalling, either by binding of wnt ligand to frizzled receptors or through mutation of the adenomatous polyposis coli (APC) and β-catenin genes is a universal defect in colon tumours and also occur at lower frequency in other tumours, such as melanoma, resulting in activation of transcription from promoters containing Tcf binding sites.

Most importantly and advantageously, the present inventors have made it possible to target and kill tumour cells with a parvovirus encoding only viral proteins, whose expression is specifically regulated by transcription factors preferentially or exclusively activated in tumour cells. Preferred virus encodes a full set of viral proteins and does not require the insertion of transgenes encoding cytotoxic proteins to facilitate tumour cell killing. Moreover the inventors have demonstrated that inserting tumour specific transcription factor binding sites does not interfere with decapsidation or conversion of the viral DNA to the double-stranded form.

Thus according to a first aspect of the present invention there is provided a viral DNA construct encoding for a parvovirus that is capable of replication in a human or animal tumour cell type wherein the construct comprises one or more selected transcription factor binding sites operatively positioned such as to promote expression of open reading frames encoding non-structural viral proteins, wherein the selected transcription factor binding sites are for a transcription factor the level or activity of which is increased in a human or animal tumour cell relative to that of a normal human or animal cell of the same type, e.g. a normal colon cell versus a colon tumor cell.

Non-structural proteins, as discussed above, are parvovirus proteins that are essential for viral replication such as autonomous parvovirus NS1 and NS2 or the Rep proteins of AAV. In a preferred construct of first aspect of the invention there is provided a viral DNA construct encoding for an autonomous parvovirus capable of replication in a human or animal tumour cell wherein the construct comprises one or more transcription factor binding sites operatively positioned within the P4 promoter such as to promote expression of non-structural (NS) proteins in the presence of said transcription factor, the level or activity of which factor being increased in a human or animal tumour cell relative to that of a normal human or animal cell of the same type.

Preferably the viral DNA construct of the first aspect has a nucleic acid sequence corresponding to that of a wild type parvovirus sequence wherein part of the wild type P4 promoter site is replaced by the one or more selected transcription factor binding sites.

As discussed above there are a number of different transcription factors that are tumour specific, i.e. whose activity or level is specifically increased by causal oncogenic mutations. Preferably the viral DNA constructs of the first aspect, and viruses encoded thereby, contain Tcf transcription factor binding sites in operational relationship with the non-structural gene (NS) open reading frames. More preferably the Tcf binding sites replace one or more wild type transcription factor binding sites in the P4 promoter. The inventors have shown that these constructs encode viruses that are selective for tumour cells containing oncogenic APC and β-catenin mutations. Thus preferred transcription factor binding sites are selectively activated in tumour cells containing oncogenic APC and β-catenin mutations.

Inserting one or more Tcf binding sites into the P4 promoter, to produce a Tcf-P4 promoter, is one embodiment of the present invention which ensures tumour selective expression of parvovirus genes. In addition to the intrinsic oncotropism of parvoviruses such as H1, parvoviruses having Tcf binding sites thus have an additional mechanism to ensure that they can only replicate selectively in cells with activated wnt signalling. The inventors have found that the parvoviruses with Tcf-P4 promoters showed levels of DNA replication comparable or higher than wild type virus in colon cancer cells. In contrast, the Tcf-P4 viruses all gave 100-fold less viral DNA than the wild type virus in normal cells. Thus the chance that these viruses will replicate in normal tissues is remote, particularly as the few normal cells with active wnt signalling, like colon crypt stem cells and early T cells, are unlikely to fulfil the other requirements for parvovirus replication.

The inventors have found that the Tcf-P4 promoter which combines the lowest basal activity with the largest inducibility has the Tcf sites inserted nearest the TATA box. Preferred such replacement sites are single or multiples of the Tcf binding sequence, e.g. containing 1 to 10, more preferably 2 to 4, most conveniently, 2 or 3 Tcf sites.

Particular Tcf sites are of consensus sequence (A/T)(A/T)CAA(A/T)GG, see Roose, J., and Clevers, H. (1999 Biochim Biophys Acta 1424, M23-37), but are more preferably as shown in the examples herein.

The constructs with the Tcf sites positioned in the reverse orientation, i.e. with the C/T rich strand of the Tcf sites on the viral coding (anti-genomic strand), showed the highest absolute activity. Thus, preferably constructs of the first aspect comprise one or more Tcf sites in the reverse orientation.

Thus a further separate aspect of the invention provides viral DNA constructs encoding for a parvovirus that is capable of replication in a human or animal tumour cell comprising one or more Tcf sites in the reverse orientation, operatively positioned such as to promote expression of open reading frames encoding non-structural viral proteins

It will be apparent that other tumour specific transcription factor binding sites may be selected to provide viral constructs that can be used to produce therapeutic parvoviruses suitable for treating different tumour types. For example, preferred tumour specific transcription factor binding sites selected from those known in the prior art such as RBPJκ, Gli-1, HIF1alpha and telomerase promoter binding sites, may be used to produce selective transcription of parvovirus non-structural genes in tumour cells whilst, repressing transcription of these genes in normal cells.

The present inventors have found that a parvovirus with Tcf sites in place of the E2F site had a normal burst size on SW480 cells, a colon cancer cell line, showing that the Tcf sites can substitute for the normally essential E2F and transactivate the promoter in the context of the virus. Replacement of the E2F may enhance the cytotoxic effects of the virus by forcing it to express NS1 even in cells in G0/G1. Thus, preferably the viral DNA construct of the first aspect has a nucleic acid sequence from which the wild type E2F enhancer is deleted. Removal of the E2F site has an additional advantage, it should severely attenuate the virus in non-colon cells.

The inventors have also found that inserting Tcf sites into P4 appears to partially compensate for deletion of the wild type Sp1 site (mut24), but this virus was the least active, consistent with previous data showing that this site is important for basal promoter activity. Thus, preferably, the viral DNA construct of the first aspect comprises a nucleic acid sequence that includes a Sp1 site.

More preferably the viral DNA construct of the present invention includes a combination of Tcf and ets sites. This is a preferred construct for targetting colon tumours, as these tumours typically have activated wnt and ras signalling pathways and ets is the transcription factor binding site for the ras pathway, (see Bos, J. L. et al. 1987, Prevalence of ras gene mutations in human colorectal cancers. Nature. 327:293-7.).

The viral construct of the first aspect preferably comprises one or more Tcf binding sites, more preferably two to ten, still more preferably two to four Tcf sites in tandem. Preferably the viral construct of the first aspect retains one or more ITRs.

Unlike adenoviruses, parvoviruses are extremely stable in the environment. This is thought to be an adaptation to the simple lifestyle of the virus: without sophisticated tools to manipulate the host organism, the virus is forced to wait long periods before accidentally infecting a new host. It is not a desirable property of a therapeutic virus and so it is advantageous to reduce capsid stability. Thus the viral DNA construct of the first aspect of the present invention preferably comprises a mutation which destabilises the capsid such as to reduce viral persistence in the environment, more preferably the mutation reduces viral persistence in the environment and broadens the tropism to include more tumour cell lines. Selection of mutants with this phenotype is relatively easy given the strong block imposed in some cells.

The present inventors have produced viral constructs and viruses encoded thereby that are very selective for cancer cells but that are relatively attenuated. It may be desirable to increase tumour specific cell killing, for example by increasing DNA replication in tumour cells and/or increasing expression of toxic viral genes such as NS1 compared with wild type parvovirus. Thus a further embodiment of the first aspect comprises a viral construct comprising mutations that increase viral replication compared with wild type and more preferably also enhance cancer cell killing in comparison to wild type virus.

To produce a tightly regulated tumour specific transcription factor driven virus, a modified promoter containing selected transcription factor binding sites, such as Tcf sites, needs to be installed. To effect this, in a preferred embodiment of the present invention the inventors have substituted part of the parvovirus P4 promoter for a tumour specific promoter, preferably comprising Tcf binding sites. More preferably the E2F enhancer is deleted from its wild type P4 location, in part or in full, more preferably completely.

Tumour specific promoter-dependent transcription, e.g. with Tcf sites, may be inhibited or repressed by basal levels of Tcf present in normal cells but transcription is preferably increased, compared with wild type parvovirus, in colon cancer cells or other cancer cells with activated wnt signalling.

This strategy contrasts with prior art parvovirus constructs, described in U.S. Pat. No. 5,585,254 (Maxwell) which propose an autonomous parvovirus which is modified by removing P4 and inserting a promoter such as PRE to control expression of the non-structural genes.

Preferred colon tumour specific parvoviruses are encoded by viral DNA constructs corresponding to the DNA sequence of MVM or Hi, preferably from a hybrid of MVM/H1, more preferably a hybrid comprising the MVM left hairpin and P4 promoter in the H1 genome.

Advantageously the use of the constructs of the invention, particularly in the form of viruses encoded thereby, to treat neoplasms such as liver metastasis is relatively non-toxic compared to chemotherapy, providing good spread of virus within the liver aided by effective replication.

Preferred tumour specific transcription factor binding sites that are used in place of wild type sites include those described above, for example, Tcf-4, HIF1alpha, RBPJκ, ets and Gli-1 sites, and a fragment of the telomerase promoter conferring tumour-specific transcription.

A most preferred transcription factor binding site is that which binds Tcf-4, such as described by Vogelstein et al in U.S. Pat. No. 5,851,775 and is responsive to the heterodimeric β-catenin/Tcf-4 transcription factor. As such the transcription factor binding site increases transcription of genes in response to increased β-catenin levels caused by APC or β-catenin mutations. The telomerase promoter is described by Wu K J. et al (1999, Nat Genet 21, 220-4) and Cong Y S. et al (1999 HumMol Genet 8, 137-42). A further preferred binding site is that of HIF1alpha, as described by Maxwell P H. et al, (1999 Nature 399, 271-5). One may use a HIF1alpha-regulated virus to target the hypoxic regions of tumours, involving no mutation of the pathway as this is the normal physiological response to hypoxia, or the same virus may be used to target cells with VHL mutations either in the familial VHL cancer syndrome, or in sporadic renal cell carcinomas, which also have VHL mutations. A retrovirus using the HIF promoter to target hypoxia in ischemia has already been described by Boast K. et al (1999 Hum Gene Ther 10, 2197-208).

Particularly the inventors have now provided viral DNA constructs, and viruses encoded thereby, which contain the Tcf transcription factor binding sites referred to above in operational relationship with the NS open reading frame open reading frames described above, particularly in place of wild type transcription factor binding sites in their promoters and shown that these are selective for tumour cells containing oncogenic APC and β-catenin mutations. Tcf-4 and its heterodimer bind to a site designated Tcf herein.

A preferred group of viral constructs of the invention are those having the further selected transcription factor binding site in a function relationship with the NS orfs.

A second aspect of the invention provides viruses comprising or encoded by the DNA constructs described above.

Having produced a virus with one or more levels of regulation to prevent or terminate replication in normal cells, it is may be advantageous to improve the efficiency of infection at the level of receptor binding. The normal cellular receptor for autonomous (H1) parvovirus is unknown. If the receptor is poorly expressed on some colon tumour cells it would be possible to restore infectivity by modifying the VP genes encoding the capsid proteins. For example, the insertion of poly-lysine (e.g. 7-20 lysine residues) could be used to target heparan sulphate proteoglycans, which are ubiquitously expressed. Insertion of the peptides NGR, PRP or RGD may also be used to target tumour endothelium or tumour cells.

In a third aspect is provided a viral DNA construct, or a virus, of the invention for use in therapy, particularly therapy of patients having neoplasms.

In a fourth aspect is provided a viral DNA construct, or a virus, of the invention in the manufacture of a medicament for the treatment of neoplasms.

In a fifth aspect of the present invention there are provided compositions comprising the viral DNA construct of the invention, particularly in the form of a virus encoded thereby, together with a physiologically acceptable carrier. Particularly compositions are characterised in that they are sterile and pyrogen free with the exception of the presence of the viral construct or virus encoded thereby. For example the carrier may be a physiologically acceptable saline.

In a sixth aspect there is provided a method of manufacture of a viral DNA construct or a virus encoded thereby, as provided by the invention characterised in that it comprises transforming an parvovirus viral genome having one or more wild type transcription factor binding sites, preferably within the P4 promoter region, controlling transcription of a non-structural gene, such as to replace one or more of these by a tumour specific transcription factor binding site.

In a seventh aspect of the present invention there is provided a method for treating a patient suffering from a neoplasm wherein a viral DNA construct or virus of the invention is caused to infect tissues of the patient, including or restricted to those of the neoplasm, and allowed to replicate such that neoplasm cells are caused to be killed.

In an eighth aspect of the invention there is provided the viral DNA construct of the invention, particularly in the form of a virus encoded thereby, for use in therapy, particularly in therapy of patients having neoplasms, e.g. malignant tumours, particularly colorectal tumours and most particularly colorectal metastases. Most preferably the therapy is for liver tumours that are metastases of colorectal tumours.

In a ninth aspect of the invention there is provided a method of manufacture of the viral DNA construct of the invention, particularly in the form of a virus encoded thereby, comprising transforming a viral genomic DNA of an autonomous parvovirus, having wild type transcription factor binding sites, particularly as defined for the first aspect, such as to operationally replace these sites by tumour specific transcription factor binding sites, particularly replacing them by Tcf transcription factor binding sites. Operational replacement may involve partial or complete deletion of the wild type site

The present invention further attempts to improve current intra-arterial hepatic chemotherapy by prior administration of a colon-targeting replicating parvovirus. DNA damaging and antimetabolic chemotherapy is known to sensitise tumour cells to replicating adenovirus in animal models (Heise et al., 1997). Such a technique may be applicable to the parvovirus of the present invention. For example, during the first cycle the present recombinant parvovirus may be administered alone, in order to determine toxicity and safety. For the second and subsequent cycles recombinant parvovirus may be administered with concomitant chemotherapy. Safety and efficacy is preferably evaluated and then compared to the first cycle response, the patient acting as his or her own control.

Route of administration may vary according to the patients needs and may be by any of the routes described for viruses such as described in U.S. Pat. No. 5,698,443 column 6, incorporated herein by reference. Suitable doses for replicating viruses of the invention are in theory capable of being very low. For example they may be of the order of from 10² to 10¹³, more preferably 10⁴ to 10¹¹, with multiplicities of infection generally in the range 0.001 to 100.

For treatment a hepatic artery catheter, e.g. a port-a-cath, is preferably implanted. This procedure is well established, and hepatic catheters are regularly placed for local hepatic chemotherapy for ocular melanoma and colon cancer patients. A baseline biopsy may be taken during surgery.

A typical therapy regime might comprise the following:

Cycle 1: parvovirus construct administration diluted in 100 ml saline through the hepatic artery catheter, on days 1, 2 and 3.

Cycle 2 (day 29): parvovirus construct administration on days 1, 2, and 3 with concomitant administration of FUDR 0.3 mg/kg/d as continuous infusion for 14 days, via a standard portable infusion pump (e.g. Pharmacia or Melody), repeated every 4 weeks.

Toxicity of viral agent, and thus suitable dose, may be determined by Standard phase I dose escalation of the viral inoculum in a cohort of three patients. If grade III/IV toxicity occurs in one patient, enrolment is continued at the current dose level for a total of six patients. Grade III/V toxicity in ≧50% of the patients determines dose limiting toxicity (DLT), and the dose level below is considered the maximally tolerated dose (MTD) and may be further explored in phase II trials.

It will be realised that GMP grade virus is used where regulatory approval is required.

It will be realised by those skilled in the art that the administration of therapeutic parvoviruses may be accompanied by inflammation and or other adverse immunological event which can be associated with e.g. cytokine release. Some viruses according to the invention may also provoke this. It will be realised that such viruses may have advantageous anti-tumour activity over at least some of those lacking this adverse effect. In this event it is appropriate that an immuno-suppressive, anti-inflammatory or otherwise anti-cytokine medication is administered in conjunction with the virus, e.g., pre-, post- or during viral administration. Typical of such medicaments are steroids, e.g., prednisolone or dexamethasone, or anti-TNF agents such as anti-TNF antibodies or soluble TNF receptor, with suitable dosage regimes being similar to those used in autoimmune therapies. For example, see doses of steroid given for treating rheumatoid arthritis (see WO93/07899) or multiple sclerosis (WO93/10817), both of which in so far as they have US equivalent applications are incorporated herein by reference.

The present invention will now be described by way of illustration only by reference to the following non-limiting Examples, Methods, Sequences and Figures. Further embodiments falling within the scope of the claims will occur to those skilled in the art in the light of these.

### FIGURES

FIG. 1

Western blot showing expression of ΔN-β-catenin (lower band) in cMM1 cells only after removal of tetracycline from the medium. The higher band corresponds to endogenous β-catenin.

FIG. 2

Promoter map showing part of the P4 promoter andthe position of the Tcf sites. The Tcf insertions (4 Tcf sites) at positions 22, 23 and 24 replace the E2F, ets and Sp1 sites. The Tcf oligo was inserted in both possible orientations. The vMM viruses have the C/T-rich strand of the Tcf sites on the viral coding (anti-genomic) strand.

FIG. 3

Basal activity of the P4-Tcf promoters determined by luciferase assays in cMM1 cells in the presence of tetracycline. “Tcf−>” indicates plasmids with the C/T-rich strand on the genomic strand; “Tcf<−” indicates plasmids with the C/T-rich strand on the coding strand.

FIG. 4

Luciferase assay in cMM1 cells showing fold induction of the P4-Tcf promoters expressed as the ratio of maximal to basal activity (−/+ tetracycline). “Tcf−>” indicates plasmids with the C/T-rich strand on the genomic strand; “Tcf<−” indicates plasmids with the C/T-rich strand on the coding strand.

FIG. 5

Western blot showing expression (at 24 hrs post infection) of NS1 and ΔN-β-catenin in cMM1 cells infected with phH1, vMM66 or vMM74 in the presence or absence of tetracycline. Upper panel: NS1 expression; lower panel: ΔN-β-catenin expression.

FIG. 5A

Western blot showing expression (at 24 hrs post infection) of NS1 and ΔN-β-catenin in 293T and cR2 cells (293T cell derivatives expressing) infected with phH1, vMM66 or vMM74. Upper panel: NS1 expression; lower panel: expression.

FIG. 6

Burst assay measuring virus production 48 Hh post infection with phH1, vMM66 and vMM74 in cMM1 cells, expressed as the ratio of virus production in the absence and presence of ΔN-β-catenin.

FIG. 7

Western blot showing expression of NS1 in the colon cancer cell line SW480 (upper panel) and the lung cancer cell line H1299 (lower panel).

FIG. 8

Viral DNA content 24 h post infection of SW480 and H1299, determined by quantitative PCR. The values are normalized to the parental phH1 results.

FIG. 9

Burst assay in SW480 and H1299. The values are normalized to phH1.

FIG. 10

Western blot showing expression of NS1 in the colon cancer cell lines Isrecol and Co115 (upper and middle panel, respectively), and in HeLa cells (lower panel).

FIG. 11

Burst assay in Isrecol, Co115 and HeLa. The values are normalized to phH1.

FIG. 12

Table listing virus constructs with 4 Tcf sites and showing the site and orientation of Tcf sites within the P4 promoter sequence.

FIG. 13

Cytopathic Effect (CPE) assay on SW480, Isrecol, Co115 and H1299 infected with phH1, vMM66 or vMM74. Cells were stained with crystal violet 8 days post infection. The m.o.i. (multiplicity of infection) is expressed as genome copies/cell, based on the HT29 titer.

FIG. 14A

Luciferase assay showing repression of Tcf-P4 promoters by dominant negative Tcf-4 (ΔN-Tcf-4). 293T cells were transfected with pTOPFLASH or P4 luciferase reporters, mutant β-catenin expression vector (β-cat Δ45S) and 50, 100 or 200 ng of ΔN-Tcf-4 expression vector.

FIG. 14B

Luciferase assay showing repression of Tcf-P4 promoters by dominant negative Tcf-4 (ΔN-Tcf-4). SW480 cells were transfected with P4 luciferase reporters and 500 ng of ΔN-Tcf-4 expression vector.

FIG. 15A

Promoter map showing part of the P4 promoter and the position of the Tcf sites. The Tcf insertions (2 Tcf sites) at positions 22, 23 and 24 replace the E2F, ets and Sp1 sites. The Tcf oligo was inserted in both possible orientations. The vMM viruses have the C/T-rich strand of the Tcf sites on the viral coding (anti-genomic) strand.

FIG. 15B

A western blot performed on LS174T L8 colon cancer cells, which are a derivative of LS174T colon cancer cells that express a dominant-negative Tcf-4 in the presence of doxycycline.

FIG. 16A

Western blots 24 h post infection showing expression of NS1 by phH1 and the viruses containing 2 Tcf sites in NB324K and HeLa cell lines for NS1.

FIG. 16B

Burst assays in HeLa and NB324K cells showing a 10- to 100-fold reduction in virus production for the viruses with 2 Tcf sites compared to phH1. (The “x” represents a missing data point.)

FIG. 17A

Western blots 24 hr post infection, showing expression of NS1 from the viruses with 2 Tcf sites and phH1, in the colon cancer cell lines SW480, Isrecol, Co115, Hct116 and HT29.

FIG. 17B

Burst assays in a panel of colon cancer cell lines. Compared with the results obtained with for the viruses with 4 Tcf sites, the viruses with 2 Tcf sites are more active in most of the cell lines tested. NS1 expression is at wild type level in Co115, a cell line that was non-permissive for all the viruses with 4 Tcf sites, except vMM66. The burst assay results show some correlation with the western blots, except for Isrecol. This may be due to the different time points used for the two assays (24 h post-infection for the western blots and 48 h for the burst assays).

FIG. 18

MTT assay results for cell lines SW480, Col5 and NB324K infected with parental virus or vMM106, which has 2 Tcf sites (in the Ets binding site of P4). Cell viability is expressed as the percentage of living cells normalised to mock infected cells.

### MATERIALS AND METHODS

Cell lines.

HT29 and 293T cells were supplied by ATCC. Isrecol (41) and SW480 cells (also available from ATCC) were provided by Dr B Sordat (Swiss Institute for Experimental Cancer Research, Epalinges, Switzerland). Isrecol and Co115 are colon tumor cell lines with high and intermediate Tcf activity, respectively (unpublished data). HeLa cells were supplied by the Imperial Cancer Research Fund cell production lab. The H1299-derived cell line H24 containing the tet-VP16 transactivator was provided by Dr C Prives (6).

CR2 cells were derived from 293T cells by infection with a lentivirus expressing myc-tagged ΔN-β-catenin (18, 26, 37).

cMM1 cells were derived from H24 cells by stable transfection with pMM1, which contains a myc-tagged ΔN-β-catenin cDNA (37) cloned into pUHD10-3 (19). H24 cells were cotransfected with 10 μg of pMM1 and 1 μg of pBabe-puro (25) and grown in medium containing 2 μg/ml puromycin and 1 μg/ml tetracycline. Transformants were single-cell cloned and screened for inducible ΔN-β-catenin expression by western-blot and in situ staining using the anti-myc antibody 9E10 (15) and the anti-β-catenin antibody C19220 (Transduction Laboratories). ΔN-β-catenin expression was induced by removing tetracycline from the medium.

Parvovirus mutagenesis.

The P4xLuc series of plasmids has been described previously by Deleu et al. in Mol Cell Biol. 18:409-19, incorporated herein by reference (10). P4mut25Luc was cut with PmeI and self-ligated to eliminate a duplication of the NcoI-AflIII fragment within the P4 promoter. P4mut22Luc, P4mut23Luc and P4mut24Luc contain a BglII linker which replaces the E2F, ets and Sp1 sites, respectively. P4mut19Luc and P4mut25Luc contain the linker immediately before the E2F and after the Sp1 sites, respectively. A double stranded oligonucleotide containing four Tcf binding sites (GATC-TCCTTTGATCTTAATCCCTTT GATCTGGATCCCTTTGATCTCCAACCCTTT-GATC) was cloned in both orientations into the BglII site of the P4xLuc plasmids to give plasmids pMM23 & 24 (mut19), pMM25 & 26 (mut22), pMM27 & 28 (mut23), pMM29 & 30 (mut24), and pMM33 & 34 (mut25), where pMM23, 25, 27, 29 and 33 have the C/T-rich strand of the Tcf site on the viral genomic strand and the rest have the Tcf sites reversed. The P4-Tcf promoters obtained were inserted into phH1, a plasmid containing the MVM left hairpin and P4 promoter in the H1 genome (21). To achieve this, the 3298 bp NdeI-EcoRI fragment of phH1 was blunted and self-ligated to give pMM39. The AflIII site at position 3147 in pMM39 was destroyed by self-ligation. The 240 bp AflIII-NcoI fragment of the P4-Tcf-Luc plasmids was cloned into the AflIII-NcoI sites of pMM39 to give pMM41 to 50. Finally, the 2338 bp NheI-SphI fragment of phH1 was cloned into the NheI-SphI sites of pMM41 to 50 to give pMM65 to 74.

To construct the virus vectors containing 2 Tcf sites, a BamHI/BglII digestion was performed on pMM42, 44, 46, 48, 50, in which the P4 promoter contains 4 Tcf sites in the reverse orientation (namely, with the C/T-rich strand of the Tcf sites on the anti-genomic strand). pMM99 to 103 were obtained by self-ligation of the 3360 bp BamHI/BglII fragment. The 2820 bp SpeI-SspI fragment of pMM99 to 103 was inserted into the 4565 bp SpeI-SspI sites of phH1, to give pMM104 to 108, the plasmids used to make viruses vMM104 to 108.

Parvovirus amplification and titration.

Virus was produced by cotransfection of phH1-derived plasmids and pXNS1 (28) into cR2 cells. Three days post-transfection cells were harvested by scraping, washed once in PBS and resuspended in 50 mM Tris, 0.5 mM EDTA pH 8.7. Virus was released by five rounds of freeze/thawing, cell debris was pelleted by centrifugation and the virus-containing supernatant was stored at 4° C.

Viral titres were estimated by measuring the amount of viral DNA in HT29 cells 24 hours after infection in the presence of hydroxyurea. This approach was used to avoid underestimating the titre by plaque assay or infectious centre assay on non-permissive indicator cells such as NB324K. None of the viruses replicate in HT29 cells in the presence of hydroxyurea. The amount of viral DNA was measured by quantitative PCR.

The sequence of parental phH1 virus (21), i.e. HI genome with MVM P4 promoter and MVM left hairpin is shown in the sequence listing herein.

Quantitative PCR assays.

Cells were harvested and DNA extraction was performed using a Dneasy Tissue kit (Qiagen) according to the manufacturer's instructions. 10 ng of DNA was used per quantitative PCR reaction. TaqMan PCR was performed using a TaqMan Universal PCR Master Mix (Perkin-Elmer), 800 nM primers (Invitrogen) and 500 nM TaqMan probe (MWG) on a PE5700 PCR machine (Perkin-Elmer). The primers and probes lie in the NS1 coding sequence: forward primer, CCACACTCAAAGAGTTGGTACATAA; reverse primer, CACCTGGTTGAGC CATCAT; probe, AACTGTCTGGCTGCATCATCATCCA.

Luciferase assays.

cMM1 cells were seeded at 3.5×10⁵ cells per 35-mm well 24 h before transfection in medium plus or minus 1 μg/ml tetracycline. Cells were lipofected (Invitrogen) for 18 h with 100 ng of reporter plasmid and 1 ng of control Renilla luciferase plasmid (Promega, Madison, Wis.). Cells were harvested 48 h later and dual luciferase reporter assays were performed according to the manufacturer's instructions (Promega) using a Biocounter (Lumac bv, Landgraaf, The Netherlands). Each value is the mean of two independent experiments and transfection efficiency is normalized to the activity of the Renilla control.

Western blotting.

Cells were infected for one hour in serum-free DMEM, after which the medium was replaced with DMEM containing 10 % FBS (Invitrogen). Cells were harvested 24 h later. NS1 expression was detected with SP8 rabbit polyclonal antibody (3, 16). The myc-tagged ΔN-β-catenin was detected with anti-β-catenin antibody C19220 (Transduction Laboratories).

Virus replication assay.

Cells were infected for one hour in serum-free DMEM, after which the medium was replaced with DMEM containing 10 % FBS (Invitrogen). Cells were harvested 48 h later and lysed by five cycles of freeze-thawing. The viral titre in the supernatant was tested on HT29 as described above.

### EXAMPLE 1

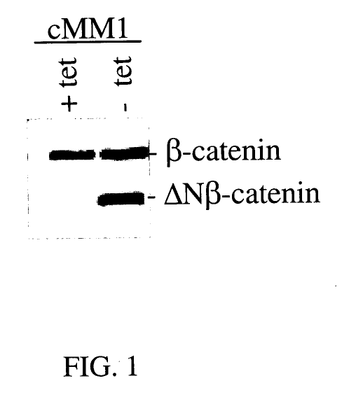

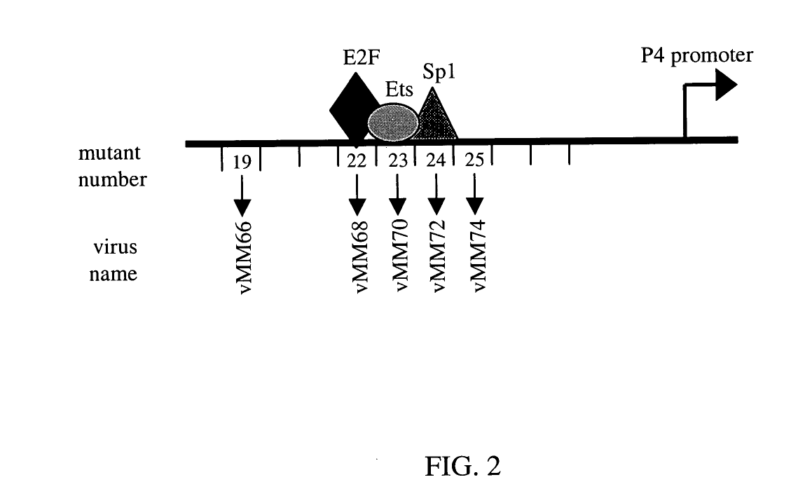

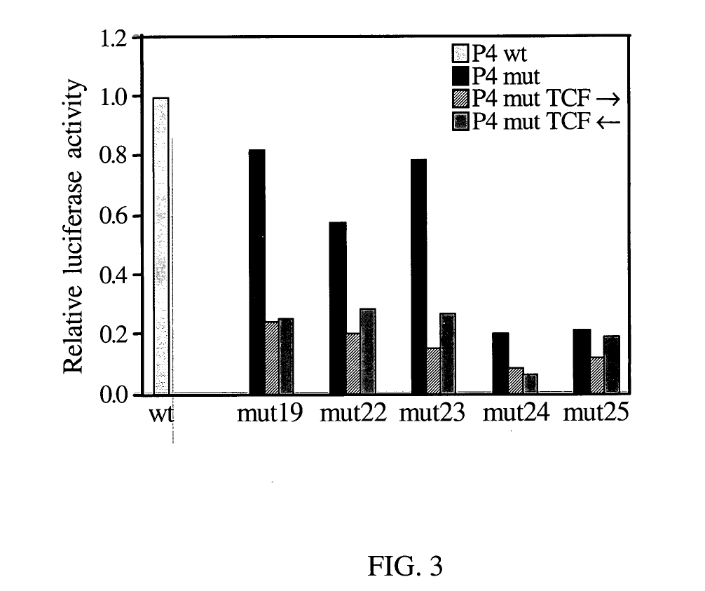

The P4 promoter has previously been characterized by BglII linker scanning mutagenesis in a hybrid MVM/H1 construct (10). We inserted four Tcf binding sites into the BglII linker of plasmids with mutations outside the hairpin (FIG. 2). The first mutant nucleotide in mut19 is 12 nucleotides downstream of the Ns1 nick site required for DNA replication. To test the inducibility of this promoter by the wnt pathway, a cell line was constructed (cMM1) that expresses an active β-catenin mutant (ΔN-β-catenin) from a tetracycline regulated promoter. The N-terminal deletion in the mutant removes the destruction box. Western blotting shows that the cMM1 cell line expresses the mutant β-catenin only after removal of tetracycline from the medium (FIG. 1). The upper band is endogenous β-catenin, the lower band is the exogenous mutant. Despite equal expression of the two forms in the induced state, only the exogenous protein can activate transcription of Tcf target genes because the endogenous protein is trapped in adherens junctions. Transfection of P4-luciferase reporters into this cell line in the presence of tetracycline tests the basal activity of the Tcf-P4 promoters (FIG. 3). Deletion of the Sp1 site (mut 24) or insertion of a BglII site immediately after the Sp1 site (mut25) reduced the luciferase activity to about 20% of the parental promoter activity. BglII insertion alone is mut 19, 22 and 23 had smaller effects. Addition of Tcf sites reduced the activity of the promoter to about 20% of the parental activity even when all of the normal binding sites were present and further reduced the already low acitivity of the mut24 promoter. This shows that the corepressors recruited by Tcf can overcome the activity of the other transcription factors present at the promoter.

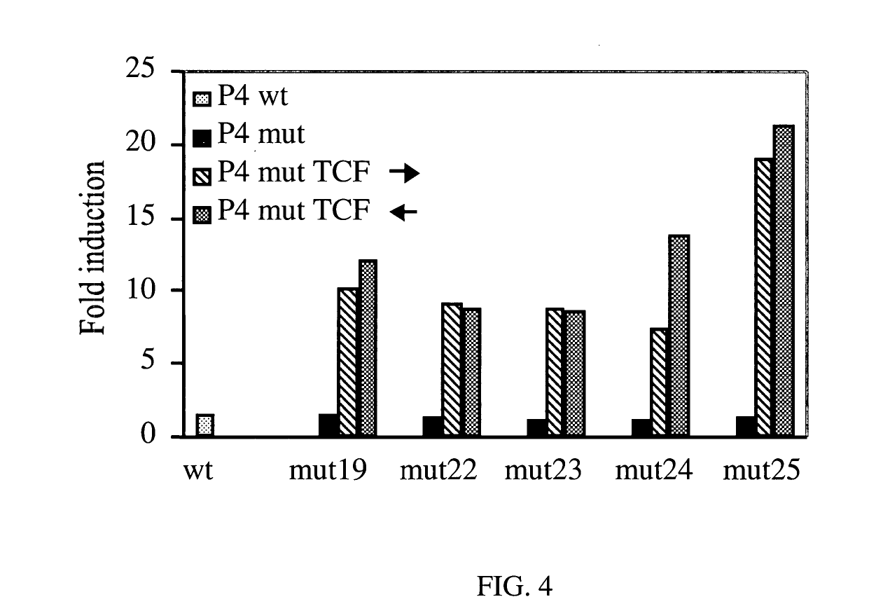

Induction of ΔN-β-catenin expression by tetracycline removal activated the Tcf-P4 promoters 10 to 20-fold (FIG. 4). The Tcf-P4 promoter which combines the lowest basal activity with the largest inducibility has the Tcf sites inserted nearest the TATA box (mutant 25). As expected, cotransfection of NS1 activated transcription from all of the promoters, regardless of β-catenin level (data not shown). The constructs with the C/T-rich strand of the Tcf sites on the coding strand were selected for further study because they gave the highest absolute activity (FIGS. 3 & 4).

### EXAMPLE 2

The Tcf promoters were transferred to a viral vector (phH1) (21) for production of virus. The viruses are called vMM66, 68, 70, 72 and 74 (FIG. 2). The packaging cell line (293T) was transduced with a lentivirus expressing ΔN-β-catenin to activate Tcf-dependent transcription. To further reduce the risk of selecting suppressors, the phH1 vectors were cotransfected with a plasmid expressing NS1 (pXNS1 (30)). Normal indicator cell lines used for infectious center assays and plaque assays are not permissive for the Tcf viruses. To titer the Tcf viruses, HT29 cells were infected in the presence of hydroxyurea and DNA was harvested after 24 hours. The number of copies of viral DNA was then measured by quantitative PCR. None of the viruses replicates in HT29 cells in the presence of hydroxyurea. Preliminary tests with wild type virus showed that the viral DNA content was the same after 24 hours in the presence of hydroxyurea as after 4 hours in the absence of hydroxyurea, suggesting that the assay measures unreplicated DNA that is stably associated with the cell in a nuclease-resistant form. For wild type virus, a titer of 100 genome copies/ml on HT29 cells corresponds to a titre of 1 pfu/ml on NB324K cells.

### EXAMPLE 3

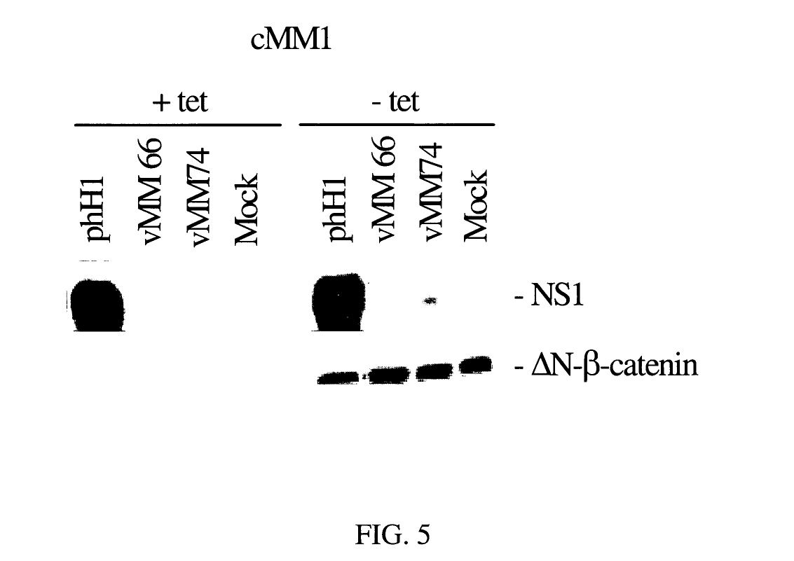

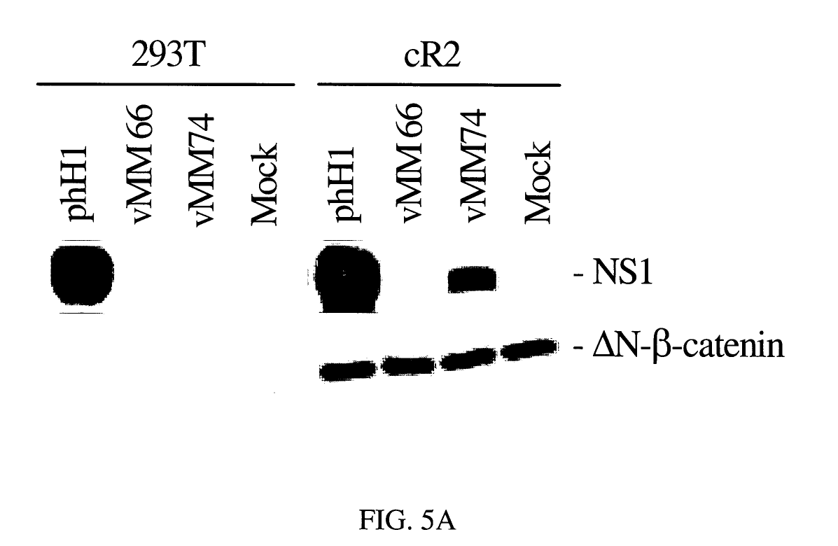

To determine whether insertion of Tcf sites into the P4 promoter can confer responsiveness to the wnt pathway in the context of the virus, cMM1 cells were infected with parental virus, vMM64 and vMM74 (the Tcf versions of mutants 19 and 25 respectively) in the presence or absence of tetracycline (FIG. 5). Induction of β-catenin expression resulted in an increase in NS1 expression from vMM74, although not to wild type levels, showing that the Tcf-P4 promoter can respond to activation of the wnt signaling pathway in the context of the virus. This is consistent with previous studies showing that heterologous genes can be expressed from modified P4 promoters in recombinant LuIII parvoviruses (25, 26).

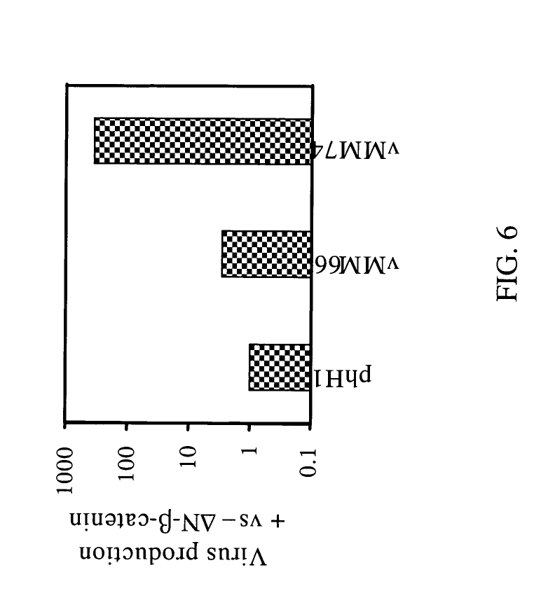

As a further test of the Tcf-P4 response to artificial activation of the wnt pathway, 293T cells and ΔN-β-catenin-expressing derivatives (cR2 cells) were infected with vMM66 and 74. Neither Tcf virus gave detectable NS1 expression in parental 293T cells, whereas expression could again be detected from vMM74 in cR2 cells (FIG. 5A). In parental 293T cells, vMM74 virus production was reduced 4,000-fold compared to phH1. Consistent with the increase in NS1 expression, there was a 300-fold increase in vMM74 virus production in the cells expressing mutant β-catenin (FIG. 6).

### EXAMPLE 4

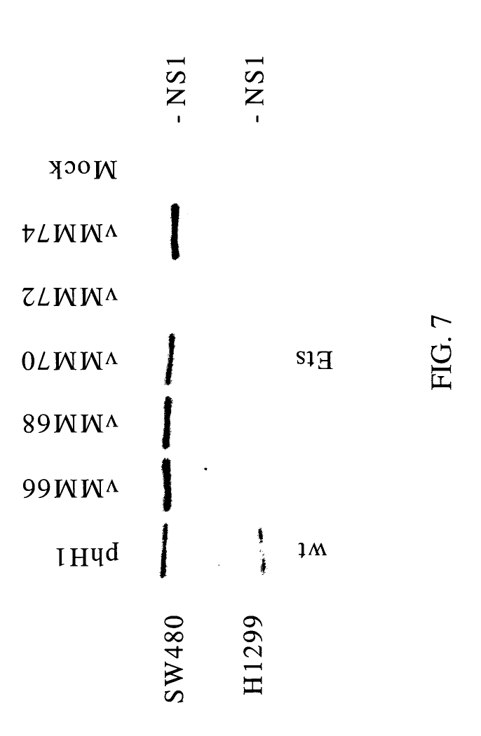

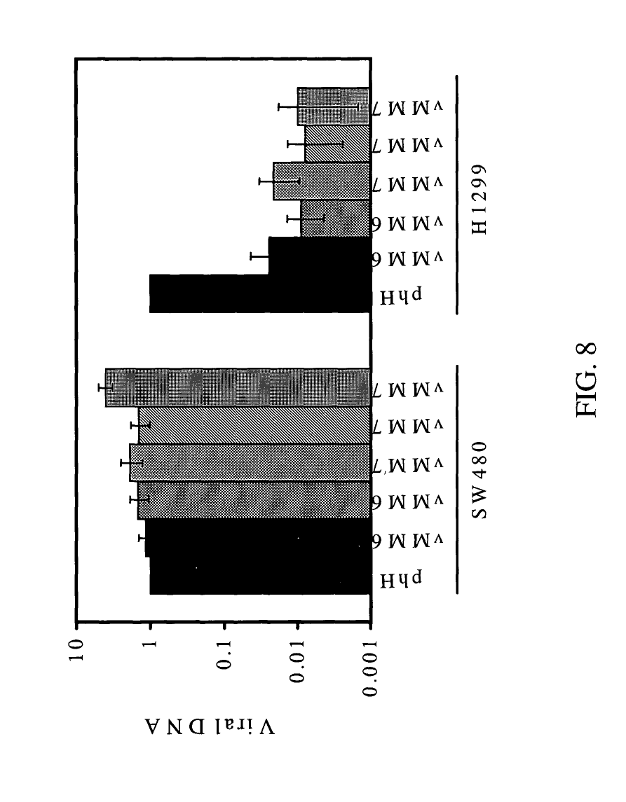

Western blotting 24 hours after infection of SW480 colon cancer cells, in which the wnt pathway is activated by mutation of APC, showed that NS1 is expressed normally from all of the Tcf viruses except vMM72, the virus with Tcf sites replacing the Sp1 site (FIG. 7 upper panel). Deletion of the Sp1 site is known to reduce the activity of the basal P4 promoter (10). Normal NS1 expression from the other Tcf-mutant viruses demonstrates that the Tcf mutations do not interfere with decapsidation or conversion of the viral DNA to the monomeric replicative form (mRF) in these cells. H1299 lung cancer cells were used as negative controls because the wnt pathway is inactive in these cells. In H1299 cells, the phH1 parental virus was able to express NS1, but all of the Tcf viruses were defective in NS1 expression (FIG. 7 lower panel). This suggests that the insertion of Tcf sites into the P4 promoter may confer selectivity for colon cancer cells, as expected from the luciferase assays. To determine whether the level of regulation of NS1 expression is sufficient to modulate viral DNA replication, the amount of DNA in cells 24 hours after infection was measured by quantitative PCR (FIG. 8). All of the Tcf viruses gave wild type levels of DNA replication in SW480 cells. In contrast, the Tcf viruses gave 100-fold less viral DNA than the parental virus in H1299 cells.

### EXAMPLE 5

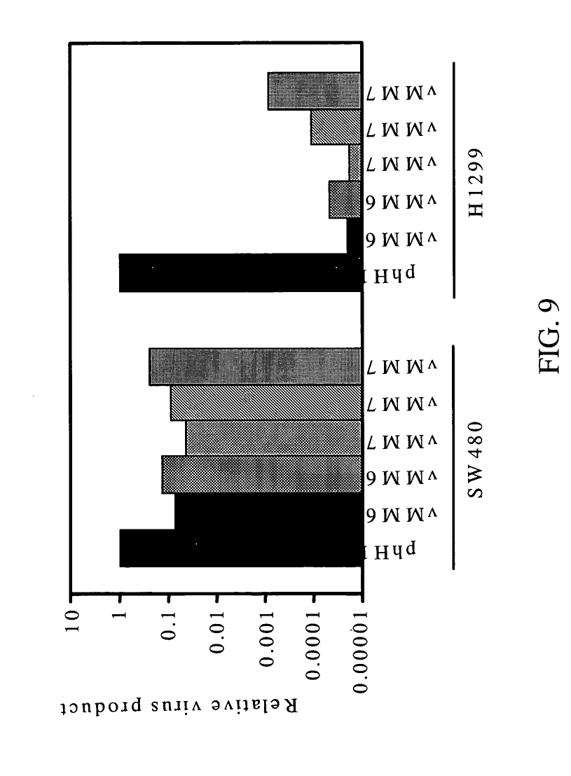

To test whether actual virus is produced from the replicated DNA, burst assays were performed (FIG. 9). Cells were infected with an amount of virus defined by HT29 assay, and the amount of virus produced was calculated by HT29 assay. The ratio of the two gives the burst size. The burst size of the Tcf-mutant viruses was nearly normal in SW480 cells, but 1000-fold reduced in H1299, 293T, NB324K and HeLa cells, which have inactive wnt signalling pathways. To test whether the effect was due to activation of the wnt pathway rather than a non-specific difference between the two cell lines, cMM1 cells were infected with vMM74 in the presence or absence of tetracycline. Compared to parental phH1 virus, vMM74 showed a large increase in burst assays following expression of the β-catenin mutant (FIG. 6). The small increase in phH1 replication following ΔN-β-catenin expression could be due to an increase in S-phase fraction caused by the oncogene. We conclude that the insertion of Tcf sites in the P4 promoter modifies the host range of the virus, and most likely confers colon cell line and wnt pathway-specific viral replication.

### EXAMPLE 6

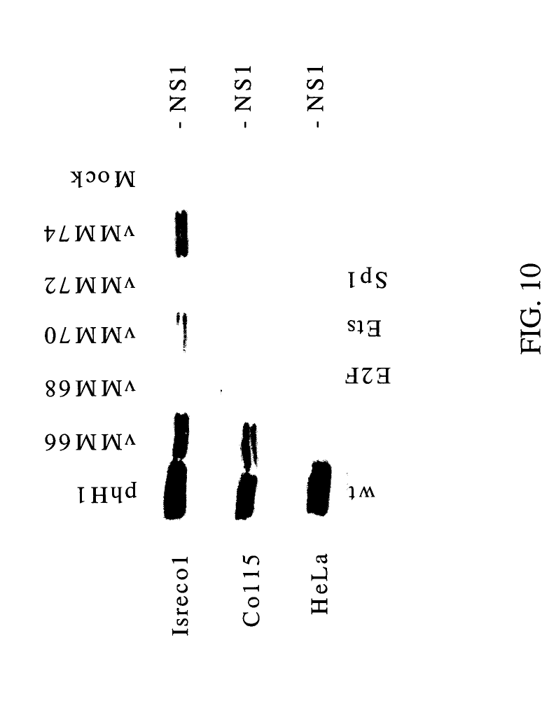

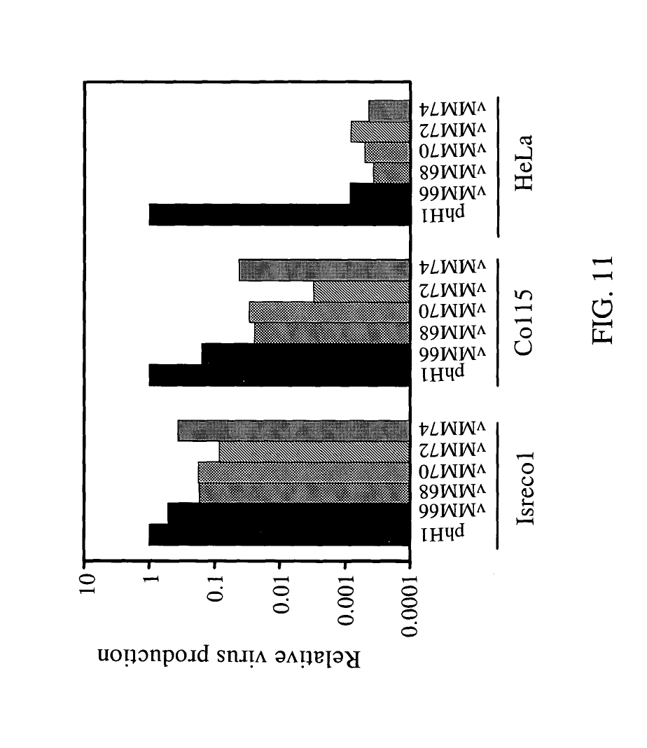

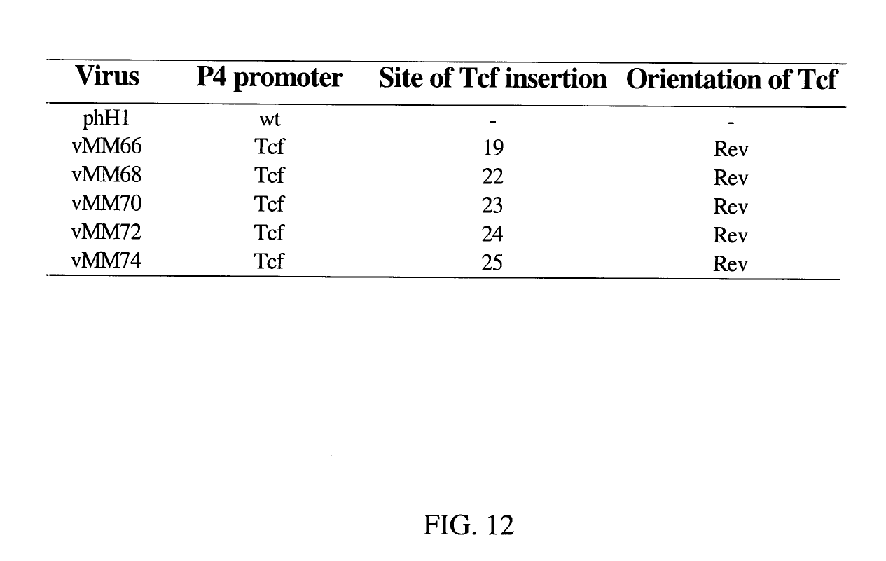

To determine whether the Tcf-regulated parvoviruses replicate in different colon cell lines, a panel of cell lines was tested. Isrecol has relatively high wnt activity and is permissive to Tcf-regulated adenoviruses. Only the parvoviruses with the Tcf sites in positions 19 and 25 (vMM66 and 74) expressed NS1 well and had a near normal burst size in Isrecol (FIG. 10 and FIG. 11). Col5 is semi-permissive for Tcf-regulated adenovirus replication. Only vMM66 was able to express NS1 in this cell line (FIG. 10). The burst size of all the Tcf-mutant viruses was reduced in Co115, ranging from a 10-fold reduction with vMM66 to a 300-fold reduction with vMM72, in which the Tcf site replaces the Sp1 site. These differences were all smaller than the 3000-fold reduction in burst size in HeLa cells, in which the wnt pathway is inactive (FIG. 11).

### EXAMPLE 7

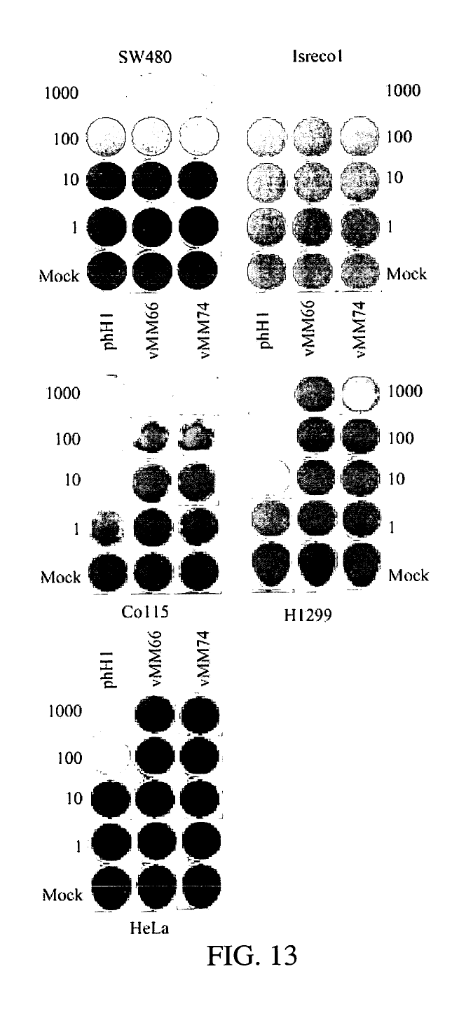

To determine the toxicity of the viruses, we performed cytopathic effect (CPE) assays on a panel of cell lines. H1299 lung cancer cells and SW480, Isrecol and Co115 colon cancer cells were infected with serial 10-fold dilutions of virus. There were substantial differences in the sensitivity of the cell lines to the parental virus (FIG. 13, phH1). In SW480 and Isrecol, the Tcf-mutant viruses vMM66 and vMM74 were as toxic as phH1 (FIG. 13, upper panels). phH1 was 100-fold more active than the Tcf-mutant viruses in Co115 (FIG. 13, lower left panel). H1299 lung cancer cells were sensitive to phH1 (FIG. 13, lower right panel), but resistant to vMM66 and vMM74. (ΔN-β-catenin) from a tetracycline regulated promoter. The N-terminal deletion in the mutant removes the destruction box. Western blotting shows that the cMM1 cell line expresses the mutant β-catenin only after removal of tetracycline from the medium (FIG. 1). The upper band is endogenous β-catenin, the lower band is the exogenous mutant. Despite equal expression of the two forms in the induced state, only the exogenous protein can activate transcription of Tcf target genes because the endogenous protein is trapped in adherens junctions. β-catenin, the lower band is the constitutively active mutant.

### EXAMPLE 8

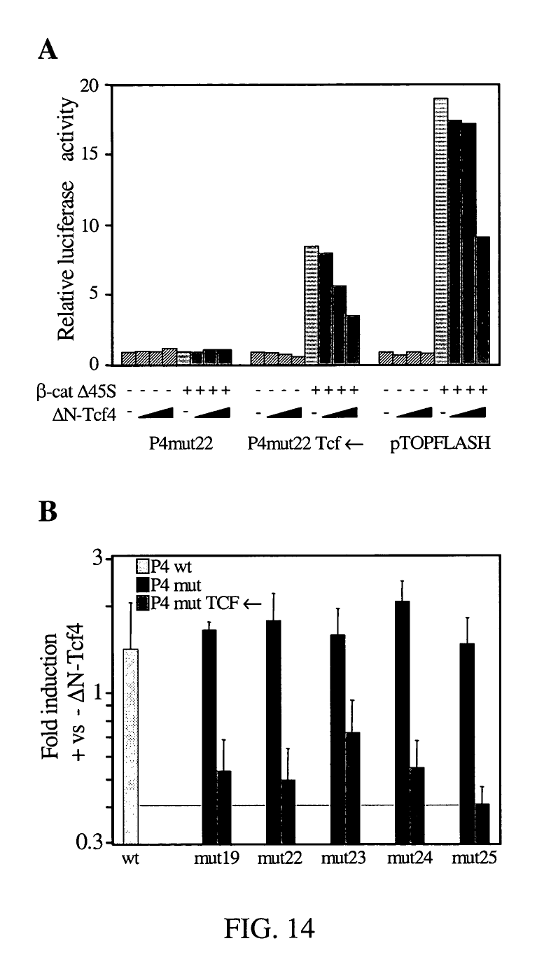

To demonstrate that the Tcf binding sites confer responsiveness to Tcf, 293T cells were co-transfected with luciferase reporters, a constitutively active β-catenin mutant (Δ45S) and increasing amounts of a dominant negative Tcf-4 mutant (ΔN-Tcf-4) (FIG. 14A). The ΔN-Tcf-4 mutant lacks the amino-terminal β-catenin binding site but can still bind to Groucho and CtBP (33). The pTOPFLASH reporter, which contains multiple Tcf sites, is widely used to test activation of the wnt pathway (42). Δ45S β-catenin had no effect on the reporters lacking Tcf sites, but activated the Tcf-P4 promoter 9-fold and the pTOPFLASH reporter 19-fold (FIG. 14A). The lower inducibility of the Tcf-P4 promoter relative to pTOPFLASH may reflect the greater complexity of the Tcf-P4 promoter, which contains additional transcription factor binding sites not present in pTOPFLASH. ΔN-Tcf-4 had no effect on the P4mut promoter lacking the Tcf site, but inhibited the transactivation of Tcf reporters by β-catenin. Activation of both pTOPFLASH and the Tcf-P4 reporter by Δ45S β-catenin was reduced to a similar extent by ΔN-Tcf-4. To confirm that Tcf contributes to the activity of the Tcf-P4 promoters in colon cancer cells, SW480 cells were transfected with the luciferase reporters and the dominant negative ΔN-Tcf-4 mutant. Unlike the wild type and P4mut promoters, the Tcf-P4 promoters were repressed 2 to 4-fold by ΔN-Tcf-4 expression (FIG. 14B). Promoter activity was reduced but not abolished by ΔN-Tcf-4 in both 293T and SW480, suggesting that this Tcf-4 mutant does not act as a strong dominant negative. The luciferase assays in FIGS. 3, 4 and 14 show that insertion of Tcf sites into the parvovirus P4 promoter confers responsiveness to activation of the prototypic wnt signaling pathway.

### EXAMPLE 9

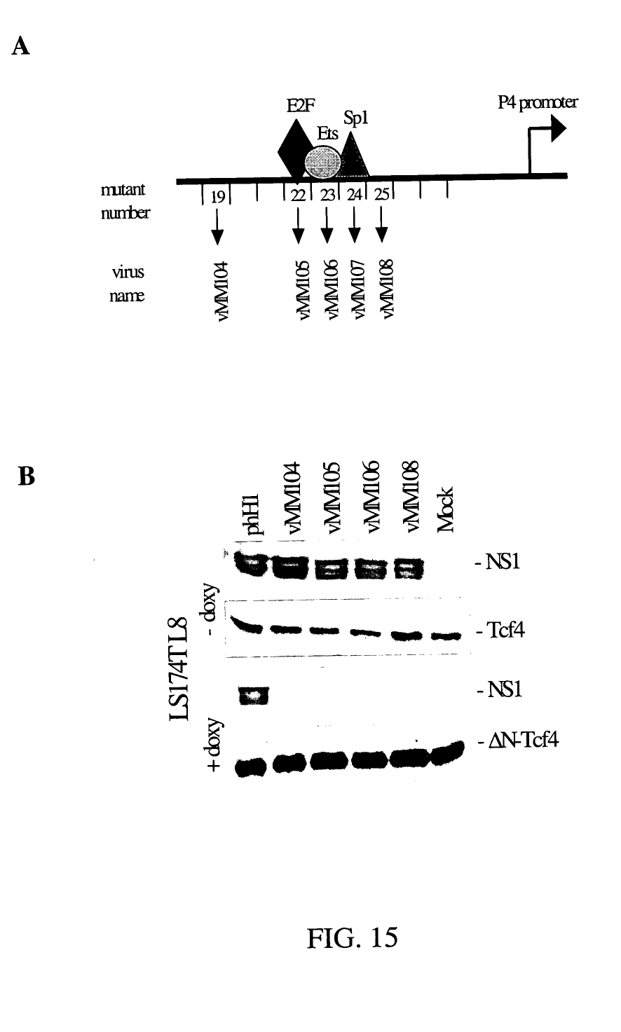

Inactivation of wnt signalling in colon cancer cells by expression of a dominant-negative Tcf inhibits replication of viruses with 2 Tcf sites in P4. A western blot (see FIG. 15B) for NS1 and Tcf-4 was performed (24 hrs post infection) on LS174T L8 colon cancer cells, which express a dominant-negative Tcf-4 when doxycycline is added to the medium. The dominant-negative Tcf-4 inhibits the active wnt signalling pathway. NS1 expression from the viruses which have 2 Tcf sites in P4 is comparable to that of wild type virus (phH1) in normal conditions (before induction of dominant negative Tcf-4). Upon induction of the dominant-negative Tcf-4 expression, the wnt signalling pathway is inhibited, and NS1 is no longer expressed from any of the viruses containing 2 Tcf sites.

### EXAMPLE 10

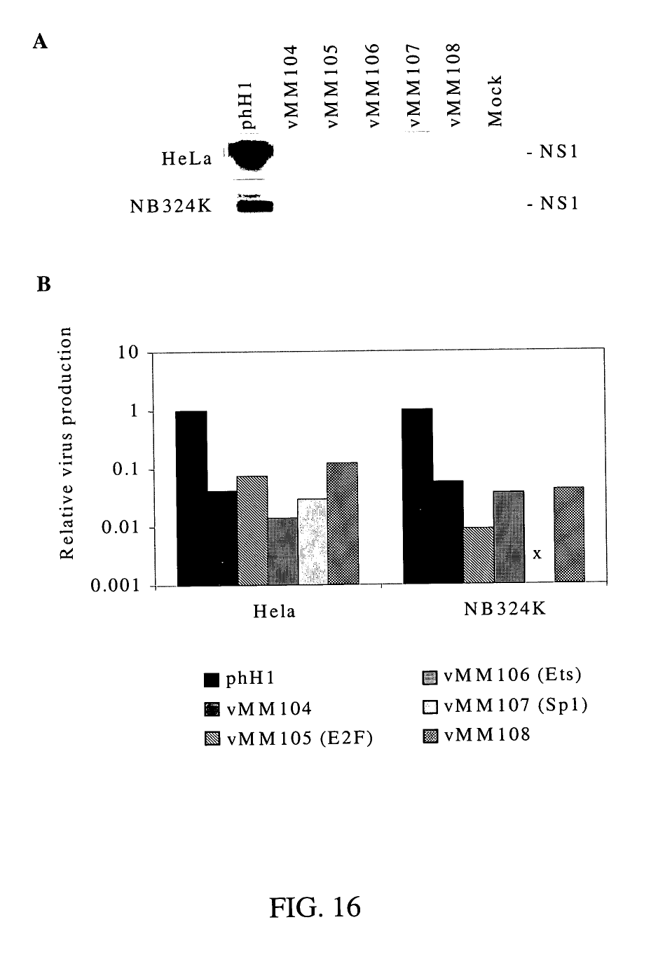

A western blot (see FIG. 16A) for NS1 was performed (24 hrs post infection) on two cell lines, i.e. HeLa and NB324K cells. These cell lines have inactive wnt signalling pathway but are transformed cells. NS1 expression is detectable for the parental phH1 virus, but not for the viruses with 2 Tcf sites. Burst assays were performed in NB324K and HeLa cells, measuring virus production at 48 hrs post infection, see FIG. 16B, which shows the values normalized to phH1. The assay shows a 10- to 100-fold reduction in production of viruses with 2 Tcf sites compared to phH1.

### EXAMPLE 11

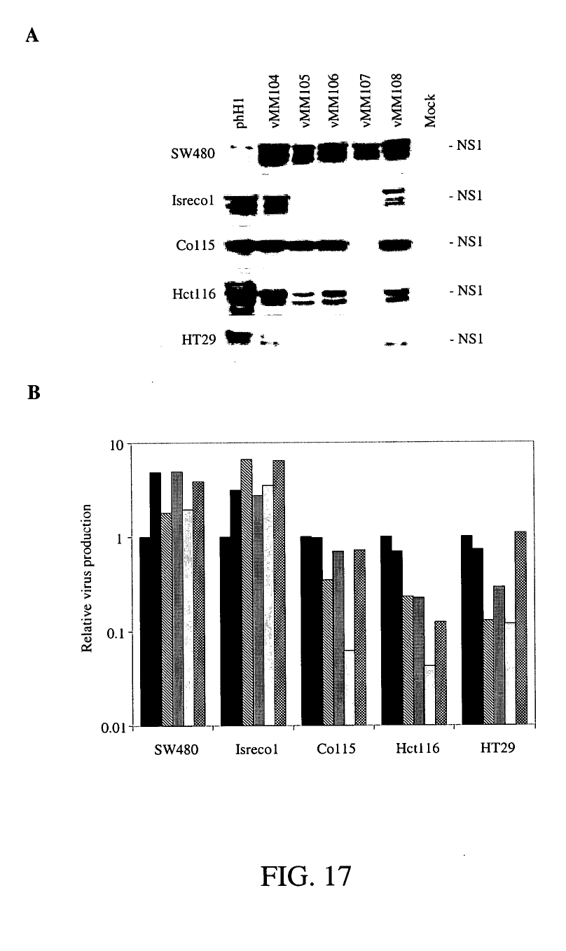

Western blots (see FIG. 17A) for NS1 were performed (24 hrs post infection) on the colon cancer cell lines SW480, Co115, Hct116 and HT29. NS1 expression is detectable for the parental phH1 virus, but not for the viruses with 2 Tcf sites.

Burst assays were performed in a panel of colon cancer cell lines, 48 h post infection with phH1 and the viruses with 2 Tcf sites, vMM104, 105, 106, 107 and 108 (see FIG. 17B). Compared with the results obtained using viruses with 4 Tcf sites, the viruses with 2 Tcf sites are more active in most of the cell lines tested. NS1 expression is wild type in Co115, a cell line that was non-permissive for all the 4 Tcf site viruses except vMM66. The burst assay results show some correlation with the western blots, except for Isrecol. This may be due to the different time points used for the two assays (24 h post-infection for the western blots and 48 h for the burst assays).

### EXAMPLE 12

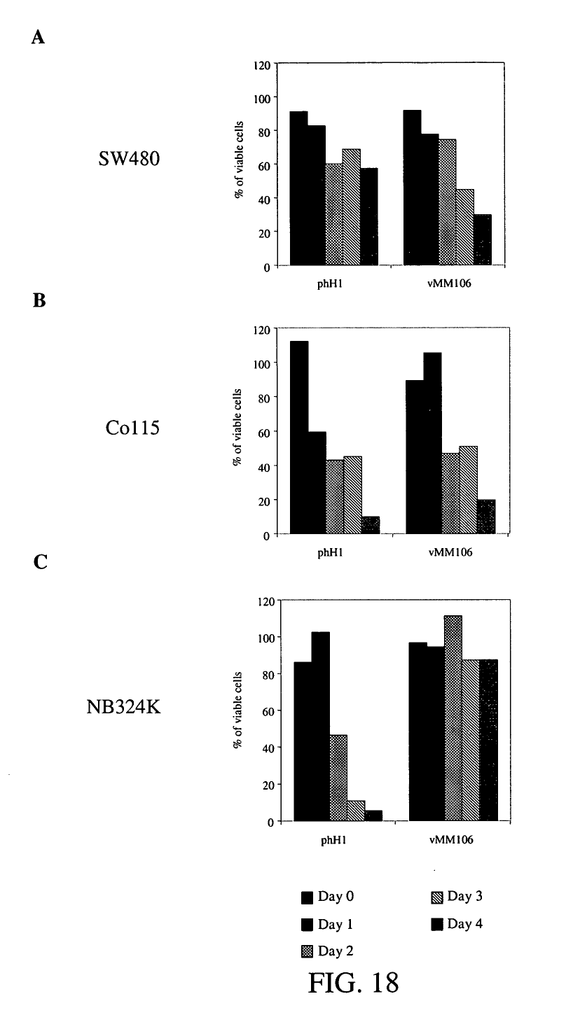

The toxicity of a virus containing 2 Tcf sites was measured by MTT assay. Results for cell lines SW480, Co115 and NB324K infected with parental virus, phH1 or vMM106 are shown in FIG. 18. Cell viability is expressed as the percentage of living cells normalized to mock infected cells. This assay expresses cell viability by measuring the metabolic activity of the cells. MTT was added to the culture medium at the indicated time points. When the cells are metabolically active, the MTT is converted to formazan by the enzymatic activity of living cells. The formazan produced was quantified by measuring absorbance at 595 nm. The results show that vMM106 (the virus with 2 Tcf sites replacing the Ets binding site in P4) is more toxic than phH1 in SW480 and as toxic as phH1 in Co115. In NB324K, (which has inactive wnt signalling), while cells are sensitive to phH1 toxicity (less than 20% of viable cells 3 days post-infection), they are not sensitive to vMM106.

### References

1. Bartkova, J., M. Thullberg, P. Slezak, E. Jaramillo, C. Rubio, L. H. Thomassen, and J. Bartek. 2001. Aberrant expression of G1-phase cell cycle regulators in flat and exophytic adenomas of the human colon. Gastroenterology. 120:1680-8.

2. Biederer, C., S. Ries, C. H. Brandts, and F. McCormick. 2002. Replication-selective viruses for cancer therapy. J Mol Med. 80:163-75.

3. Brockhaus, K., S. Plaza, D. J. Pintel, J. Rommelaere, and N. Salome. 1996. Nonstructural proteins NS2 of minute virus of mice associate in vivo with 14-3-3 protein family members. J Virol. 70:7527-34.

4. Brunori, M., M. Malerba, H. Kashiwasaki, and R. Iggo. 2001. Replicating adenoviruses that target tumors with constitutive activation of the wnt signaling pathway. J Virol. 75:2857-2865.

5. Caillet-Fauquet, P., M. Perros, A. Brandenburger, P. Spegelaere, and J. Rommelaere. 1990. Programmed killing of human cells by means of an inducible clone of parvoviral genes encoding non-structural proteins. EMBO J. 9:2989-95.

6. Chen, X., L. J. Ko, L. Jayaraman, and C. Prives. 1996. p53 levels, functional domains, and DNA damage determine the extent of the apoptotic response of tumor cells. Genes Dev. 10:2438-51.

7. Cornelis, J. J., P. Becquart, N. Duponchel, N. Salome, B. L. Avalosse, M. Namba, and J. Rommelaere. 1988. Transformation of human fibroblasts by ionizing radiation, a chemical carcinogen, or simian virus 40 correlates with an increase in susceptibility to the autonomous parvoviruses H-1 virus and minute virus of mice. J Virol. 62:1679-86.

8. Cornelis, J. J., N. Spruyt, P. Spegelaere, E. Guetta, T. Darawshi, S. F. Cotmore, J. Tal, and J. Rommelaere. 1988. Sensitization of transformed rat fibroblasts to killing by parvovirus minute virus of mice correlates with an increase in viral gene expression. J Virol. 62:3438-44.

9. Cotmore, S. F., and P. Tattersall. 1986. The NS-1 polypeptide of the autonomous parvovirus MVM is a nuclear phosphoprotein. Virus Research. 4:243-50.

10. Deleu, L., F. Fuks, D. Spitkovsky, R. Horlein, S. Faisst, and J. Rommelaere. 1998. Opposite transcriptional effects of cyclic AMP-responsive elements in confluent or p27KIP-overexpressing cells versus serum-starved or growing cells. Mol Cell Biol. 18:409-19.

11. Deleu, L., A. Pujol, S. Faisst, and J. Rommelaere. 1999. Activation of promoter P4 of the autonomous parvovirus minute virus of mice at early S phase is required for productive infection. J Virol. 73:3877-85.

12. Doerig, C., B. Hirt, P. Beard, and J. P. Antonietti. 1988. Minute virus of mice non-structural protein NS-1 is necessary and sufficient for transactivation of the viral P39 promoter. J Gen Virol. 69:2563-73.

13. Dupressoir, T., J. M. Vanacker, J. J. Cornelis, N. Duponchel, and J. Rommelaere. 1989. Inhibition by parvovirus H-1 of the formation of tumors in nude mice and colonies in vitro by transformed human mammary epithelial cells. Cancer Research. 49:3203-8.

14. Enger, P. O., F. Thorsen, P. E. Lonning, R. Bjerkvig, and F. Hoover. 2002. Adeno-associated viral vectors penetrate human solid tumor tissue in vivo more effectively than adenoviral vectors. Hum Gene Ther. 13:1115-25.

15. Evan, G. I., G. K. Lewis, G. Ramsay, and J. M. Bishop. 1985. Isolation of monoclonal antibodies specific for human c-myc proto-oncogene product. Mol Cell Biol. 5:3610-6.

16. Faisst, S., S. R. Faisst, T. Dupressoir, S. Plaza, A. Pujol, J. C. Jauniaux, S. L. Rhode, and J. Rommelaere. 1995. Isolation of a fully infectious variant of parvovirus H-1 supplanting the standard strain in human cells. J Virol. 69:4538-43.

17. Faisst, S., D. Guittard, A. Benner, J. Y. Cesbron, J. R. Schlehofer, J. Rommelaere, and T. Dupressoir. 1998. Dose-dependent regression of HeLa cell-derived tumours in SCID mice after parvovirus H-I infection. I J Cancer. 75:584-9.

18. Fuerer, C., and R. Iggo. 2002. Adenoviruses with Tcf binding sites in multiple early promoters show enhanced selectivity for tumour cells with constitutive activation of the wnt signalling pathway. Gene Ther. 9:270-81.

19. Gossen, M., and H. Bujard. 1992. Tight control of gene expression in mammalian cells by tetracycline-responsive promoters. Proc Natl Acad Sci USA. 89:5547-51.

20. Hanson, N. D., and S. L. d. Rhode. 1991. Parvovirus NS1 stimulates P4 expression by interaction with the terminal repeats and through DNA amplification. J Virol. 65:4325-33.

21. Kestler, J., B. Neeb, S. Struyf, J. Van Damme, S. F. Cotmore, A. D'Abramo, P. Tattersall, J. Rommelaere, C. Dinsart, and J. J. Cornelis. 1999. cis requirements for the efficient production of recombinant DNA vectors based on autonomous parvoviruses. Hum Gene Ther. 10:1619-32.

22. Legendre, D., and J. Rommelaere. 1994. Targeting of promoters for trans activation by a carboxy-terminal domain of the NS-1 protein of the parvovirus minute virus of mice. J Virol. 68:7974-85.

23. Lorson, C., L. R. Burger, M. Mouw, and D. J. Pintel. 1996. Efficient transactivation of the minute virus of mice P38 promoter requires upstream binding of NS1. J Virol. 70:834-42.

24. Maxwell, I. H., and F. Maxwell. 1999. Control of parvovirus DNA replication by a tetracycline-regulated repressor. Gene Ther. 6:309-13.

25. Maxwell, I. H., F. Maxwell, S. L. Rhode, 3rd, J. Corsini, and J. O. Carlson. 1993. Recombinant LuIII autonomous parvovirus as a transient transducing vector for human cells. Hum Gene Ther. 4:441-50.

26. Maxwell, I. H., A. L. Spitzer, C. J. Long, and F. Maxwell. 1996. Autonomous parvovirus transduction of a gene under control of tissue- specific or inducible promoters. Gene Ther. 3:28-36.

27. Morgenstern, J. P., and H. Land. 1990. Advanced mammalian gene transfer: high titre retroviral vectors with multiple drug selection markers and a complementary helper-free packaging cell line. Nucleic Acids Res. 18:3587-96.

28. Naldini, L., U. Blomer, P. Gallay, D. Ory, R. Mulligan, F. H. Gage, I. M. Verma, and D. Trono. 1996. In vivo gene delivery and stable transduction of nondividing cells by a lentiviral vector. Science. 272:263-7.

29. Previsani, N., S. Fontana, B. Hirt, and P. Beard. 1997. Growth of the parvovirus minute virus of mice MVMp3 in EL4 lymphocytes is restricted after cell entry and before viral DNA amplification: cell-specific differences in virus uncoating in vitro. J Virol. 71:7769-80.

30. Pujol, A., L. Deleu, J. P. Nuesch, C. Cziepluch, J. C. Jauniaux, and J. Rommelaere. 1997. Inhibition of parvovirus minute virus of mice replication by a peptide involved in the oligomerization of non-structural protein NS1. J Virol. 71:7393-403.

31. Raj, K., P. Ogston, and P. Beard. 2001. Virus-mediated killing of cells that lack p53 activity. Nature. 412:914-7.

32. Rhode, S. L. d. 1989. Both excision and replication of cloned autonomous parvovirus DNA require the NS1 (rep) protein. J Virol. 63:4249-56.

33. Roose, J., and H. Clevers. 1999. TCF transcription factors: molecular switches in carcinogenesis. Biochim Biophys Acta. 1424:M23-37.

34. Rubio, M. P., S. Guerra, and J. M. Almendral. 2001. Genome replication and postencapsidation functions mapping to the non-structural gene restrict the host range of a murine parvovirus in human cells. J Virol. 75:11573-82.

35. Takemasa, I., H. Higuchi, H. Yamamoto, M. Sekimoto, N. Tomita, S. Nakamori, R. Matoba, M. Monden, and K. Matsubara. 2001. Construction of preferential cDNA microarray specialized for human colorectal carcinoma: molecular sketch of colorectal cancer. Biochem Biophys Res Commun. 285:1244-9.

36. Tao, N., G. P. Gao, M. Parr, J. Johnston, T. Baradet, J. M. Wilson, J. Barsoum, and S. E. Fawell. 2001. Sequestration of adenoviral vector by Kupffer cells leads to a nonlinear dose response of transduction in liver. Mol Ther. 3:28-35.

37. Telerman, A., M. Tuynder, T. Dupressoir, B. Robaye, F. Sigaux, E. Shaulian, M. Oren, J. Rommelaere, and R. Amson. 1993. A model for tumor suppression using H-I parvovirus. Proc Natl Acad Sci U S A. 90:8702-6.

38. Tetsu, O., and F. McCormick. 1999. Beta-catenin regulates expression of cyclin D1 in colon carcinoma cells. Nature. 398:422-6.

39. van de Wetering, M., R. Cavallo, D. Dooijes, M. van Beest, J. van Es, J. Loureiro, A. Ypma, D. Hursh, T. Jones, A. Bejsovec, M. Peifer, M. Mortin, and H. Clevers. 1997. Armadillo coactivates transcription driven by the product of the Drosophila segment polarity gene dTCF. Cell. 88:789-99.

40. Van Pachterbeke, C., M. Tuynder, J. P. Cosyn, L. Lespagnard, D. Larsimont, and J. Rommelaere. 1993. Parvovirus H-1 inhibits growth of short-term tumor-derived but not normal mammary tissue cultures. Int J Cancer. 55:672-7.

41. Saurin J C, Nemoz-Gaillard E, Sordat B, Cuber J C, Coy D H, Abello J, Chayvialle J A. 1999. Bombesin stimulates adhesion, spreading, lamellipodia formation, and proliferation in the human colon carcinoma Isrecol cell line. Cancer Res. 1999 Feb. 15;59(4):962-7.

42. Korinek, V., N. Barker, P. J. Morin, D. van Wichen, R. de Weger, K. W. Kinzler, B. Vogelstein, and H. Clevers. 1997. Constitutive transcriptional activation by a beta-catenin-Tcf complex in APC-/- colon carcinoma [see comments]. Science 275:1784-7.

1. A viral DNA construct encoding for a parvovirus that is capable of replication in a human or animal tumour cell comprising one or more selected transcription factor binding sites operatively positioned such as to promote expression of open reading frames encoding non-structural viral proteins, wherein the selected transcription factor binding sites are for a transcription factor the level or activity of which is increased in a human or animal tumour cell relative to that of a normal human or animal cell of the same type.

2. A viral DNA construct according to claim 1 wherein the transcription factor binding sites are positioned within the P4 promoter region.

3. A viral DNA construct according to claim 1 comprising a nucleic acid sequence corresponding to that of a wild type parvovirus wherein part of the wild type P4 promoter region is replaced by the one or more selected transcription factor binding sites.

4. A viral DNA construct as claimed in claim 1 characterised in that the wild type E2F enhancer is deleted.

5. A viral DNA construct as claimed in claim 1 characterised in that the selected transcription factor binding sites are for a transcription factor whose activity or level is specifically increased by causal oncogenic mutations.

6. A viral DNA construct as claimed in claim 1 characterised in that the selected transcription factor binding site is selectively activated in tumour cells containing oncogenic APC and β-catenin mutations.

7. A viral DNA construct as claimed in claim 1 characterised in that the selected transcription factor binding sites are single or multiples of a Tcf binding site sequence.

8. A viral DNA construct as claimed in claim 1 wherein the selected transcription factor binding site is a Tcf binding site sequence in the reverse orientation.

9. A viral DNA construct as claimed in claim 1 comprising a mutation which destabilises the capsid such as to reduce viral persistence in the environment.

10. A virus comprising or encoded by a viral DNA construct claim 1.

11. A viral DNA construct, or a virus, as claimed in claim 1 for use in therapy.

12. A viral DNA construct, or a virus, as claimed in claim 10 characterised in that the therapy is of patients having neoplasms.

13. A viral construct, as claimed in claim 1 characterised in that it is capable of causing death of the tumour cell.

14. A viral DNA construct or a virus, as claimed in claim 1 wherein viral replication is reduced in normal cells compared with wild type virus.

15. A viral DNA construct or a virus, as claimed in claim 1 characterised in that viral replication is enhanced in cancer cells compared with replication of wild type virus in cancer cells.

16. Use of a viral construct, or a virus, as claimed in claim 1 in the manufacture of a medicament for the treatment of neoplasms.

17. A composition comprising a viral construct, or a virus, as claimed in claim 1 together with a physiologically acceptable carrier.

18. A composition as claimed in claim 17 characterised in that it is sterile and pyrogen free with the exception of the presence of the viral construct or virus encoded thereby.

19. A composition as claimed in claim 17 or claim 18 characterised in that the carrier is a physiologically acceptable saline.

20. A method of manufacture of a viral DNA construct or a virus encoded thereby as claimed in claim 1 characterised in that it comprises transforming a viral genome having one or more wild type transcription factor binding sites controlling transcription of non-structural open reading frames, such as to replace one or more of these by tumour specific transcription factor binding sites.

21. A method as claimed in claim 19 characterised in that the modified genome is transferred to a prokaryote for production of viral construct DNA.

22. A method of manufacture of a virus characterised in that viral construct DNA produced by a method as claimed in claim 20, is transferred to a mammalian cell for production of virus.

23. A method for treating a patient in need of therapy for a neoplasm wherein a viral DNA construct or virus as claimed in claim 1 is caused to infect tissues of the patient, including or restricted to those of the neoplasm, and allowed to replicate such that neoplasm cells are caused to be killed.

24. A method as claimed in claim 23 characterised in that the patient is in need of therapy for a colon cell derived tumour.

25. A method as claimed in claim 24 characterised in that the colon cell derived tumour is a metastasis located in the liver of the patient.


SEQUENCE LISTING (5 sequences)

SEQ ID NO: 1 - DNA, 5121 bp, Artificial Sequence
  Description of Artificial Sequence Parvovirus H1 with promoter P4 and left hairpin from MVM (phH1)
aaactccctgaaccgcttatcatttttagaactgaccaaccatgttcacgtaagtgacgtgatgacgcgcgctgcgcgcgcgccttcggacgtcacacgtcacttacgtttcacatggttggtcagttctaaaaatgataagcggttcagggagtttaaaccaaggcgcgaaaaggaagtgggcgtggtttaaagtatataagcaactactgaagtcagttacttatcttttctttcattctgtgagtcgagacgcacagaaagagagtaaccaactaaccatggctggaaatgcttactctgatgaagttttgggagcaaccaactggttaaaggaaaaaagtaaccaggaagtgttctcatttgtttttaaaaatgaaaatgttcaactgaatggaaaagatatcggatggaatagttacaaaaaagagctgcaggaggacgagctgaaatctttacaacgaggagcggaaactacttgggaccaaagcgaggacatggaatgggaaaccacagtggatgaaatgaccaaaaagcaagtattcatttttgattctttggttaaaaaatgtttatttgaagtgcttaacacaaagaatatatttcctggtgatgttaattggtttgtgcaacatgaatggggaaaagaccaaggctggcactgccatgtactaattggaggaaaggactttagtcaagctcaagggaaatggtggagaaggcaactaaatgtttactggagcagatggttggtaacagcctgtaatgtgcaactaacaccagctgaaagaattaaactaagagaaatagcagaagacaatgagtgggttactctacttacttataagcataagcaaaccaaaaaagactataccaagtgtgttctttttggaaacatgattgcttactattttttaactaaaaagaaaataagcactagtccaccaagagacggaggctattttcttagcagtgactctggctggaaaactaactttttaaaagaaggcgagcgccatctagtgagcaaactgtatactgatgagatgaaaccagaaacggtcgagaccacagtgaccactgcacaggaagctaagcgcggcagaattcaaactagagaggaggtctcgattaaaaccacactcaaagagttggtacataaaagagtaacctcaccagaagactggatgatgatgcagccagacagttacattgaaatgatggctcaaccaggtggagaaaacttgcttaaaaatacactagagatctgtacactgactctagcaagaaccaaaacagcctttgacttgattctggaaaaagctgaaaccagcaaactagccaacttttccatggctagcaccagaacctgtagaatctttgctgagcatggctggaactatattaaagtctgccatgccatctgttgtgtgctgaatagacaaggaggcaaaaggaacactgtgctctttcacggaccagccagcacaggcaaatctattattgcacaagccatagcacaagcagttggtaatgttggttgttacaatgctgccaatgtgaactttccatttaatgactgtaccaacaaaaacttgatttgggtggaagaagctggtaactttggccagcaagtaaaccaattcaaagctatttgttctggccaaaccatacgcattgatcaaaaaggaaaaggcagcaaacagattgaaccaacaccagttattatgaccaccaacgagaacattaccgtggttagaataggctgtgaggaaagaccagaacacactcaaccaatcagagacagaatgctcaacattcacctgacacgtacactacctggtgactttggtttggtggataagcacgaatggcctctgatctgtgcttggttggtgaagaatggttaccaatctaccatggcttgttactgtgctaaatggggcaaagttcctgattggtcagaggactgggcggagccgaagctagacactcctataaattcgctaggttcaatgcgctcaccatctctgactccgagaagtacgcctctcagccaaaactacgctcttactccacttgcatcggaccttgcggacctagctctagagccttggagcacaccaaatactcctgttgcgggcactgcagcaagccaaaacactggggaggctggttccacagcctgccaaggtgctcaacggagcccaacctggtccgagatcgaggcggatttgagagcttgcttcagtcaagaacagttggagagcgacttcaacgaggagctgaccttggactaaggtacaatggcacctccagctaaaagagctaaaagaggtaaggggctaagggatggttggttggtggggtactaatgtatgactacctgttttacaggcctgaaatcacttggttctaggttgggtgcctcctggctacaagtacctgggaccagggaacagccttgaccaaggagaaccaaccaacccttctgacgccgctgccaaagaacacgacgaagcctacgaccaatacatcaaatctggaaaaaatccttacctgtacttctctcctgctgatcaacgcttcattgaccaaaccaaagacgccaaggactggggcggcaaggttggtcactacttttttagaaccaagcgagcttttgcacctaagctttctactgactctgaacctggcacttctggtgtgagcagacctggtaaacgaactaaaccacctgctcacatttttgtaaatcaagccagagctaaaaaaaaacgcgcttctcttgctgcacagcagaggactctgacaatgagtgatggcaccgaaacaaaccaaccagacactggaatcgctaatgctagagttgagcgatcagctgacggaggtggaagctctgggggtgggggctctggcgggggtgggattggtgtttctactgggacttatgataatcaaacgacttataagtttttgggagatggatgggtagaaataactgcacatgcttctagacttttgcacttgggaatgcctccttcagaaaactactgccgcgtcaccgttcacaataatcaaacaacaggacacggaactaaggtaaagggaaacatggcctatgacacacatcaacaaatttggacaccatggagcttggtagatgctaatgcttggggagtttggttccaaccaagtgactggcagttcattcaaaacagcatggaatcgctgaatcttgactcattgagccaagaactatttaatgtagtagtcaaaacagtcactgaacaacaaggagctggccaagatgccattaaagtctataataatgacttgacggcctgtatgatggttgctctggatagtaacaacatactgccttacacacctgcagctcaaacatcagaaacacttggtttctacccatggaaaccaaccgcaccagctccttacagatactactttttcatgcctagacaactcagtgtaacctctagcaactctgctgaaggaactcaaatcacagacaccattggagagccacaggcactaaactctcaattttttactattgagaacaccttgcctattactctcctgcgcacaggtgatgagtttacaactggcacctacatctttaacactgacccacttaaacttactcacacatggcaaaccaacagacacttggcatgcctccaaggaataactgacctaccaacatcagatacagcaacagcatcactaactgcaaatggagacagatttggatcaacacaaacacagaatgtgaactatgtcacagaggctttgcgcaccaggcctgctcagattggcttcatgcaacctcatgacaactttgaagcaaacagaggtggcccatttaaggttccagtggtaccgctagacataacagctggcgaggaccatgatgcaaacggagccatacgatttaactatggcaaacaacatggcgaagattgggccaaacaaggagcagcaccagaaaggtacacatgggatgcaattgatagtgcagctgggagggacacagctagatgctttgtacaaagtgcaccaatatctattccaccaaaccaaaaccagatcttgcagcgagaagacgccatagctggcagaactaacatgcattatactaatgtttttaacagctatggtccacttagtgcatttcctcatccagatcccatttatccaaatggacaaatttgggacaaagaattggacctggaacacaaacctagactacacgtaactgcaccatttgtttgtaaaaacaacccaccaggtcaactatttgttcacttggggcctaatctgactgaccaatttgacccaaacagcacaactgtttctcgcattgttacatatagcactttttactggaagggtattttgaaattcaaagccaaactaagaccaaatctgacctggaatcctgtataccaagcaaccacagactctgttgccaattcttacatgaatgttaagaaatggctcccatctgcaactggcaacatgcactctgatccattgatttgtagacctgtgcctcacatgacatactaaccaaccaactatgtttctctgtttgcttcacataatacttaaactaactagactacaacataaaaatatacacttaataatagattattaaaaataacataatatggtaggttaactgtaaaaaataatagaacttttggaataaatatagttagttggttaatgttagatagaatataaaaagattttgtattttaaaataaatatagttagttggttaatgttagatagaatataaaaagattttgtatttgggaaataaaaagggtggttgggtggttggttggtactcccttagactgaatgttagggaccaaaaaaataataaaataattaaaatgaacaaggactactgtctattcagttgaccaactgaacctatagtatcactatgtttttagggtgggggggtgggagatacatacgttcgctatggaccaagtggtaccggttggttgcgctcaaccaaccggaccggcttagccggtccggttggttcgagcttagcaaccaaccggtaccacttgg

SEQ ID NO: 2 - DNA, 59 bp, Artificial Sequence
  Description of Artificial Sequence Synthetic oligonucleotide
gatctcctttgatcttaatccctttgatctggatccctttgatctccaaccctttgatc

SEQ ID NO: 3 - DNA, 25 bp, Artificial Sequence
  Description of Artificial Sequence Primer
ccacactcaaagagttggtacataa

SEQ ID NO: 4 - DNA, 19 bp, Artificial Sequence
  Description of Artificial Sequence Primer
cacctggttgagccatcat

SEQ ID NO: 5 - DNA, 25 bp, Artificial Sequence
  Description of Artificial Sequence Synthetic probe
aactgtctggctgcatcatcatcca